(19) United States
(12) Patent Application Publication
(10) Pub. No.: US 2026/0232362 A1
(43) Pub. Date: Aug. 13, 2026

(54) BALANCE COMPENSATED SCREW THREAD AND METHOD FOR MAKING THE SAME

(71) Applicant: Wayne Willert, Port Washington, NY (US)

(72) Inventor: Wayne Willert, Port Washington, NY (US)

(21) Appl. No.: 19/469,748

(22) PCT Filed: Mar. 27, 2024
(86) PCT No.: PCT/US2024/021650

§ 371 Date: Sep. 26, 2025

Related U.S. Application Data
(60) Provisional application No. 63/455,729, filed on Mar. 30, 2023.

(51) Int. Cl. A61B 17/86; B23G 1/32

(52) U.S. Cl. CPC A61B 17/866; B23G 1/32

(57) ABSTRACT

A method for making a balance compensated orthopedic screw and the end product of the method is disclosed. In one embodiment, the bar stock is selectively rotated in predetermined rotation degrees, and in opposite directions while the thread is being cut into the bar stock. In another implementation, the thread cutting blades can be selectively rotated around a stationary bar stock in a predetermined direction of rotation while cutting the thread and changing the direction of rotation of the cutting blades to impart cutting in a different direction on the bar stock.

### BACKGROUND

### Technical Field

The present invention relates to the surfaces of orthopedic screws. More particularly, it relates to a balance compensation on the surfaces of the threading for orthopedic screws.

During the manufacturing of products of any kind, including screws of any kind, machine lines or manufacturing marks are usually left behind, even on a microscopic level. When manufacturing screws, and in particular orthopedic screws, the formation of the threads by the machinery being used to form the same or tool wear behaviors and corresponding machines surfaces, in conjunction with the material from which the screw is made, can and often does result in the thread having machine lines which herein are referred to as the “grain”. These machine lines, or grain, are directional in nature as a result of the direction in which the screw threads are formed by the tools used for the same and have different heights and depths throughout the surfaces of the screw.

The grain of screws and their threads can have an impact on the ability to accurately drive/insert the screw into the bone in a precisely aligned manner. This is primarily due to the bone (or other workpiece into which the screw is driven) having a grain of its own, and the interaction between the bone grain and the thread grain (in any direction) as the screw is driven into the bone. The grain of the bone is a result of many things, including, but not limited to the various layers of bone, both hard and spongy/soft.

The manufacturing marks, machine lines or the inconsistent or erratic direction of the “grain” of the thread as referred to herein cannot generally be seen by the human eye but is readily visible on a microscopic level. This is because screw manufacturers polish, electro-plate or otherwise treat the screw to remove the grain such that it appears smooth and clean to the look and even touch. FIG. 1 shows a 12× magnified image of an existing bone screw thread. This screw has been polished by the manufacturer, yet it can be seen that there are manufacturing marks or grain lines associated with the same. FIG. 2 shows a 25× magnified image of the same screw, and it will be readily apparent from this image that there is clearly a grain associated with the thread resulting from the manufacturing of the screw thread, notwithstanding the manufacturers attempt to polish or remove the same.

As will be appreciated and mentioned above, the grain of the screw and thread has a direction that is the result of how the thread was formed in the material for the screw. In all manufacturing examples, the screw threads are cut or milled into a bar stock, and that cutting causes a grain to be formed in the direction of the cutting, milling or rolling dies. When the screw threads are rolled into the bar stock, the grain is formed in the direction of the rolling and the form used for the same. The same holds true for screws that are cast using cast molds. In other embodiments, the screw threads are grinded or compressed into the bar stock, which also causes a grain to be formed in the direction of the grinding or compression.

Examples of various factors that have an impact on the grain of a manufactured screw include, but not are exclusively limited to varying densities of the screw material used to form the screw and varying thicknesses of the screw material.

Another example relates to faceted orthopedic screws where facets are formed in the threads or on the leading or trailing edge of the threads by cutting the bar stock with a particular harmonic vibration, this cutting can result in a more enhanced grain on the facets themselves, which are formed in the direction of cutting used to form the facets. FIG. 3 shows a 25× magnified image of an exemplary orthopedic screw having facets cut into the same. FIG. 4 shows a 50× magnified image of the same screw. These images very clearly show the grain formed by forming the facets in the thread. Additionally, it will be apparent that at the transition point of the facets, the grain is offset (i.e., is not perfectly aligned with the adjacent grain of the adjacent facet) on the screw core.

Thus, it will be appreciated that when the surface grain of the screw thread (with or without facets) comes into contact with the grain of the bone during insertion, if these “grains” are not aligned with each other (even on the microscopic level), it can cause the screw to slightly mis-align from its intended direction/position during insertion within the bone for the particular application. In addition, misalignment (in conjunction with the grains on the thread surfaces) can unnecessarily cut material mass, causing a residue between the thread and the product (e.g., bone), which further causes a destabilization in the products intended mechanical performance. This destabilization can lead to, for example, infection and/or rejection of the screw implant by the patient receiving the same.

Since bone grains, like wood grains, are not regular or predictable in every instance, it is desirable to manufacture a bone screw surface that compensates for these slight misalignments resulting from the combination of the screw grain with that of the bone grain. This is achieved by the present invention by providing a balanced and aligned manufacturing or machine surface marks during the manufacture of the bone screw.

### SUMMARY

In accordance with one implementation of the present invention, the screw thread is balance compensated by changing the direction of the screw grain throughout the screw during manufacturing. This and other aspects of the invention are achieved in accordance with one embodiment by selectively controlling and changing the cutting direction of cutting tools used to form the threads during manufacturing. In accordance with another embodiment, the direction of the bar stock can be changed relative to the cutting tools to selectively control and change the cutting direction during the manufacturing process.

Other aspects and features of the present principles will become apparent from the following detailed description considered in conjunction with the accompanying drawings. It is to be understood, however, that the drawings are designed solely for purpose of illustration and not as a definition of the limits of the present principles, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings wherein like reference numerals denote similar components throughout the views:

FIG. 1 is a 12× magnified image of a bone screw according to the known prior art;

FIG. 2 is a 25× magnified image of the bone screw of FIG. 1;

FIG. 3 is a 25× magnified image of another bone screw according to the known prior art;

FIG. 4 is a 50× magnified image of the bone screw of FIG. 3;

FIGS. 5 and 6 are exemplary images of bones in partial cross-section showing the various layers of the same;

FIGS. 7A-7D are cross sectional views of a screw showing the partitioning into ½, ⅓, ¼ and ⅛ sections, respectively;

FIG. 8 is a flow diagram of the method for providing a balance compensated screw thread, according to an embodiment of the invention; and

FIGS. 9A and 9B are plan views showing the use of multiple cutting devices on the same bar stock, according to another implementation of the present invention.

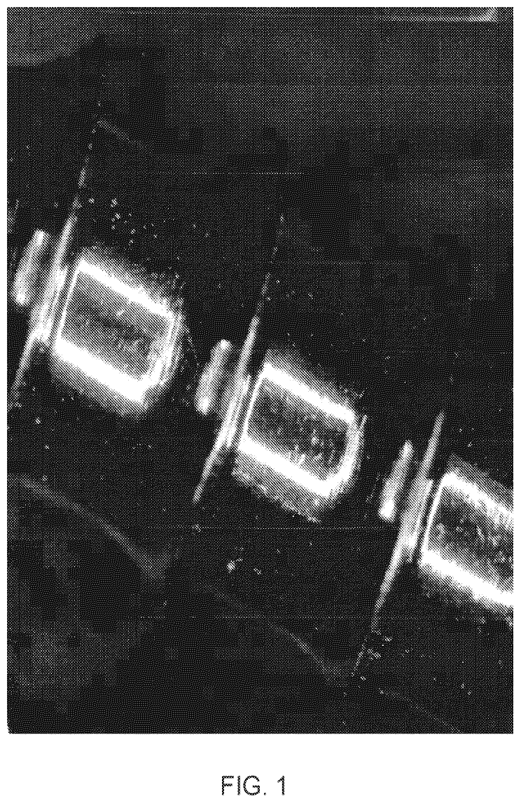

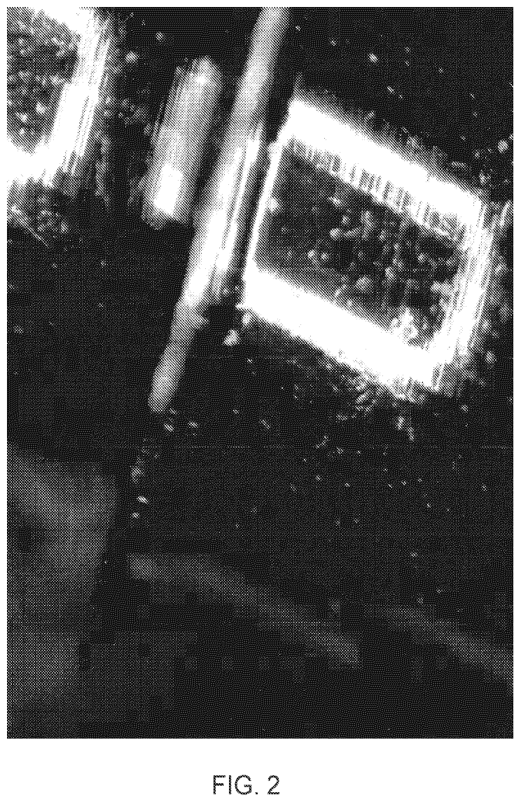

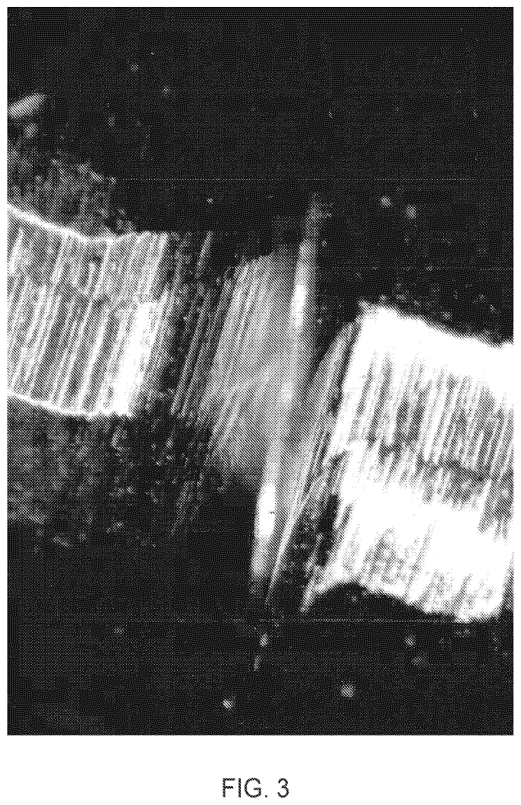

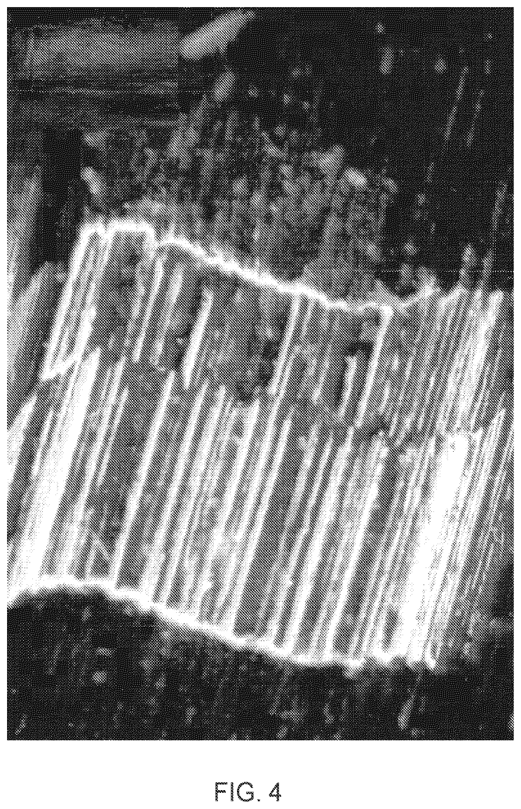

### DETAILED DESCRIPTION

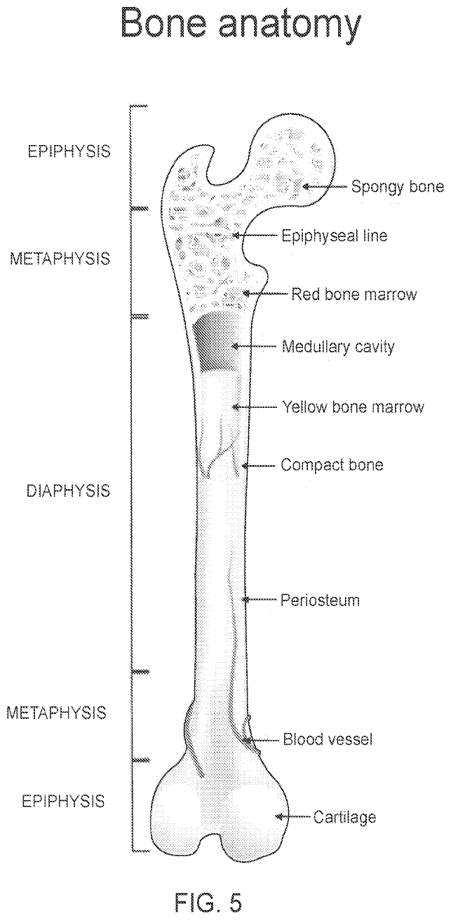

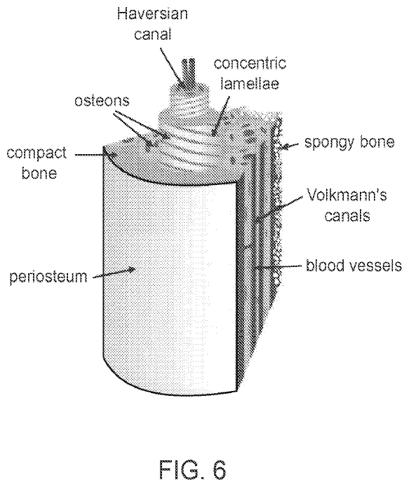

FIGS. 5 and 6 show images of bones in partial cross-section to demonstrate the various layers involved when driving a screw into the same. As will be appreciated there are many layers to bones, including, for example, the periosteum (outer most layer), compact bone, spongy bone, along with blood vessels, osteons, concentric lamellae, the Haversian canal and the Volkmann's canal. Each of these layers have varying density, and varying thickness and thus, when inserting a bone screw into the bone, it becomes apparent that such varying densities and thicknesses alone have an impact on a screw being inserted into the same. Add to this the thread grain of that bone screw and how it interacts with each of these layers, and it becomes even more apparent that each layer (of a different density and thickness) can have a different impact on the alignment of the screw as it is inserted into the bone. For example, as the screw is inserted into the bone (via a channel or pre-drilled hole), the different densities and thickness of the layers of bone can have different impact on different portions of the screw as it descends into the channel or pre-drilled hole.

The present invention proposes to change the manner in which screws are manufactured in order to provide a geometrically “balanced” threading surface such that the manufacturing marks or the grain automatically aligns with and balances the screw during insertion and thereby compensates for any possible misalignments or damage to the bone mass resulting from the same. In another alternative embodiment, the concept of “aligned balanced surface” on threading refers to “non-cutting” thread surfaces (i.e., surfaces on the angled portions of the thread from extending from the shaft to the peak of the thread) which do not cut as it is driven into the bone, thus eliminating the possible formation of residue

This is achieved, in accordance with one embodiment by cutting or forming the thread in parts or sections over the entire length of the same. For example, by dividing the screw (from a cross-section perspective) into halves, thirds, quarters, eighths, etc. we can form the thread in each geometric portion separately such that the surface grain or manufacturing marks/machine lines meet and align to counter each other throughout the entire thread to compensate deflecting misalignments caused by the workpiece grain (e.g. bone).

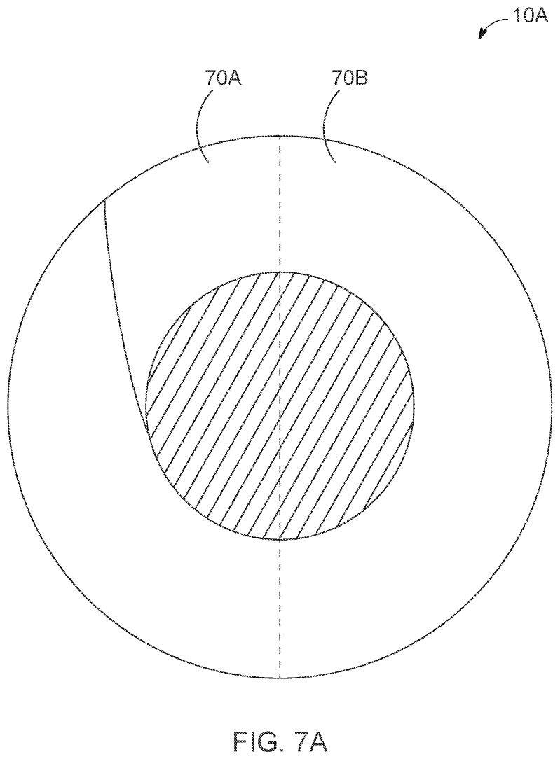

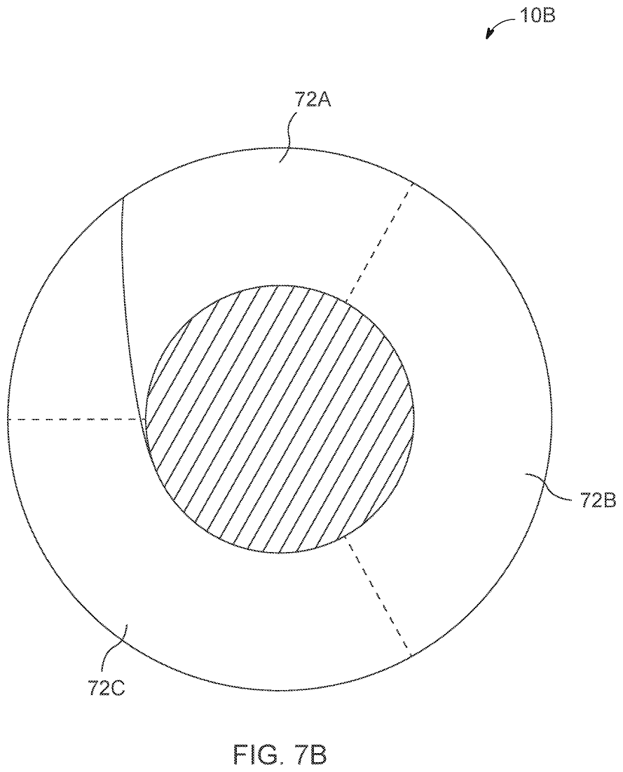

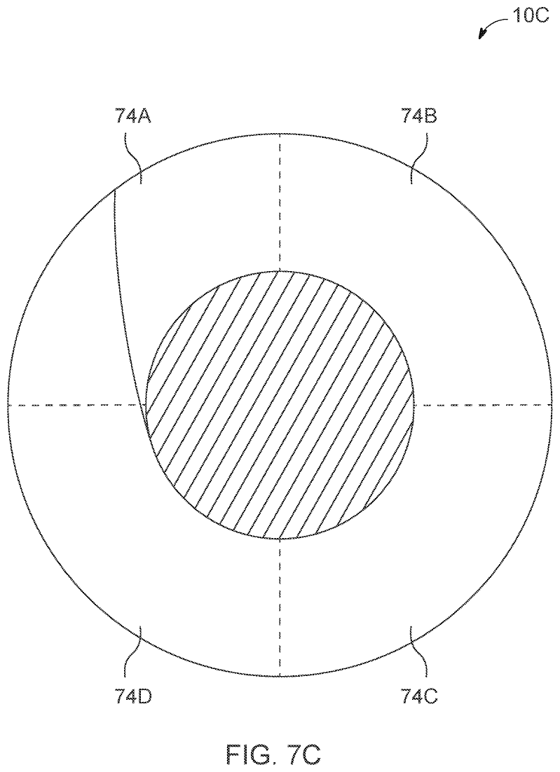

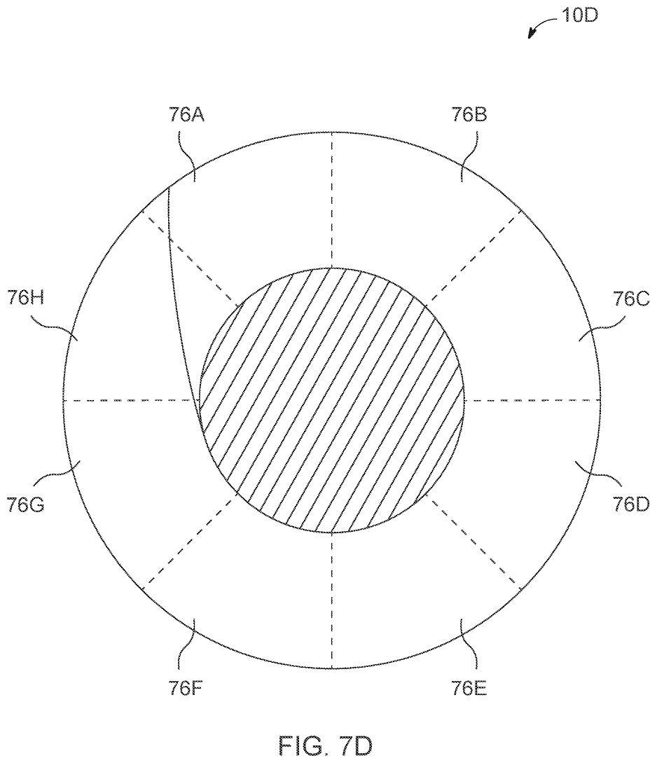

FIG. 7A shows a cross section view of a screw 10A divided into two halves 70A and 70B. FIG. 7B shows a cross section view of a screw 10B divided into thirds, 72A, 72B and 72C. FIG. 7C shows a cross section view of a screw 10C divided into quarters, 74A, 74B, 74C and 74D. FIG. 7D shows a cross section view of a screw 10D divided into eights, 76A, 76B, 76C, 76D, 76E, 76F and 76G.

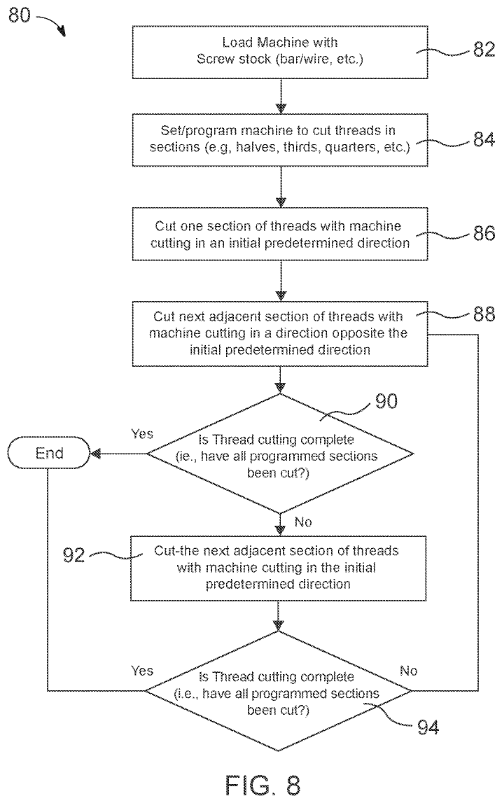

FIG. 8 shows a flow diagram of the method 80 of manufacturing a balance compensated screw thread according to one embodiment of the invention. We start with loading the machine (e.g., Computer Numeric Control-CNC) with the bar stock or wire stock from which the screw will be made (82). The machine is set or programmed (84) to cut the threads in sections as selected by the user. As noted above, this can be as simple as two sections (halves) or can be more complex with three or more sections. The machine is then started, and one section of threads are cut into the stock in an initial or predetermined first direction (86). The next adjacent section of threads is then cut into the stock in a second direction that is opposite the initial or predetermined first direction (88). A determination is then made (90) as to whether the thread cutting is complete (i.e., have all sections established in step 84 been complete). If yes, the process ends, and the screw is ejected from the machine. If no at step 90, the process continues to cut the next adjacent section of threads in the initial or predetermined first direction. A determination is then again made (94) as to whether the thread cutting is complete. If the thread cutting is complete, the process ends. If the thread cutting is not complete, the process returns to step 88, and starts over with the next adjacent section being cut at the first direction.

As will be appreciated by those of skill in the art, the direction of the cutting directions can be clockwise or counter-clockwise relative to the screw stock, so long as the opposite cutting technique is used in adjacent sections of the screw designated at the outset of the manufacturing.

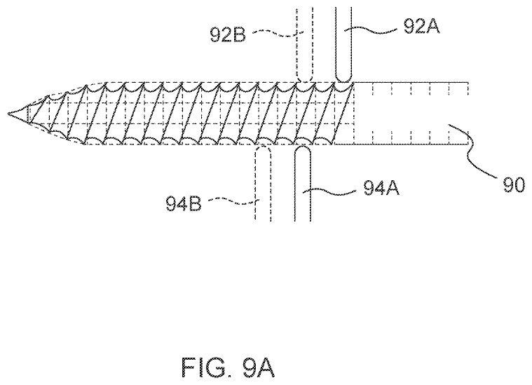

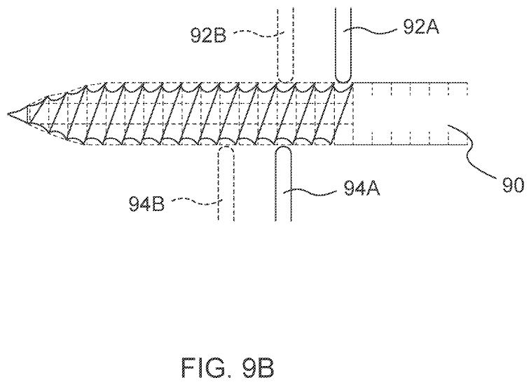

In accordance with another embodiment to provide a geometrically “balanced” threading, the present invention proposes more than one cutting tool engaged with the bar stock. Referring to FIG. 9A, there is shown an example of a bar stock 90, and a first cutting blade 92A and a second cutting blade 94A. In this embodiment, blade 92a and blade 94A will rotate in opposite directions relative to each other and are offset from each other by one thread such that, in this example, one full rotation of the bar stock 90 will cause the thread cut by blade 92A to meet up with the thread cut by blade 94A. Upon completion of one rotation, the blades 92A and 92B are moved to a next position shown by 92B and 94B to complete the next rotation of the bar stock. In this manner, each rotation of the bar stock causes two adjacent threads to be cut with opposing cutting directions. In this exemplary embodiment, two cutting blades simultaneously cutting adjacent threads is shown. It will be appreciated that modifications to the spacing of the blades and threads cut can be altered without departing from the scope of the present invention.

FIG. 9B shows another example where the blades 92A and 92B are spaced from each other such that it will take two rotations of the bar stock 90 for the thread being cut by blade 92A to meet the thread cut by blade 94A. In this manner, the change in direction of the cutting (or the grain of the screw) will occur for every two threads and not every adjacent thread as shown in the embodiment of FIG. 9A.

In accordance with another embodiment to provide a geometrically “balanced” threading, the present invention proposes the use of more than one cutting tool engaged with the bar stock during manufacturing. This would allow multiple cutting tools to rotate in different directions simultaneously during manufacturing of the screw/screw threads.

While there have been shown, described and pointed out fundamental novel features of the present principles, it will be understood that various omissions, substitutions and changes in the form and details of the methods described and devices illustrated, and in their operation, may be made by those skilled in the art without departing from the spirit of the same. For example, it is expressly intended that all combinations of those elements and/or method steps which perform substantially the same function in substantially the same way to achieve the same results are within the scope of the present principles. Moreover, it should be recognized that structures and/or elements and/or method steps shown and/or described in connection with any disclosed form or implementation of the present principles may be incorporated in any other disclosed, described or suggested form or implementation as a general matter of design choice. It is the intention, therefore, to be limited only as indicated by the scope of the claims appended hereto.

Having described preferred embodiments, which serve to illustrate various concepts, structures and techniques that are the subject of this patent, it will now become apparent to those of ordinary skill in the art that other embodiments incorporating these concepts, structures and techniques may be used. Additionally, elements of different embodiments described herein may be combined to form other embodiments not specifically set forth above.

Accordingly, it is submitted that that scope of the patent should not be limited to the described embodiments but rather should be limited only by the spirit and scope of the following claims.

1. A method for manufacturing an orthopedic screw comprising:
rotating a bar stock in a machine for a first predetermined amount of rotation in a first direction;
cutting (86) a thread into the bar stock using a blade for the first predetermined amount of rotation;
changing the first rotation direction of the bar stock to a second rotation direction opposite the first rotation direction for a second predetermined amount of rotation; and
cutting (88) the thread into the bar stock using the blade for the second predetermined amount of rotation.

2. The method according to claim 1, wherein the first predetermined amount of rotation is 45 degrees.

3. The method according to claim 1, wherein the second predetermined amount of rotation is 45 degrees.

4. The method according to claim 1, wherein the first predetermined amount of rotation is 90 degrees.

5. The method according to claim 1, wherein the second predetermined amount of rotation is 90 degrees.

6. The method according to claim 1, wherein the first predetermined amount of rotation is in a range of 90-180 degrees.

7. The method according to claim 1, wherein the second predetermined amount of rotation is in a range of 90-180 degrees.

8. An orthopedic screw manufactured in accordance with the method of claim 1.

9. A method for manufacturing an orthopedic screw comprising:
rotating a bar stock in a machine for a first predetermined amount of rotation in a first rotation direction;
cutting (86) a thread into the bar stock using a blade rotating opposite the first rotation direction for the first predetermined amount of rotation;
changing the first rotation direction of the bar stock to a second rotation direction opposite the first rotation direction for a second predetermined amount of rotation; and
cutting (88) the thread into the bar stock using the blade rotating opposite the second rotation direction for the second predetermined amount of rotation.

10. A method for manufacturing an orthopedic screw comprising:
placing a bar stock in a machine;
cutting (86) a thread into the bar stock using a first blade rotating in the first direction; and
cutting (88) the thread into the bar stock using a second blade positioned so as to not interfere with the cutting by the first blade, the second blade rotating in a second direction opposite the first direction.
